(19) United States
(12) Patent Application Publication
(10) Pub. No.: US 2011/0224663 A1
(43) Pub. Date: Sep. 15, 2011

(54) CONTROL CIRCUITRY FOR A TISSUE ABLATION SYSTEM

(75) Inventors: Warren P. Heim, Boulder, CO (US); David G. Matsuura, Encinitas, CA (US); Walter Dean Gillespie, Carlsbad, CA (US); Louise Focht, Del Mar, CA (US)

(73) Assignee: Tornier, Inc., Edina, MN (US)

(21) Appl. No.: 12/989,392

(22) PCT Filed: Apr. 23, 2009
(86) PCT No.: PCT/US09/41540

§ 371 Date: Jun. 2, 2011

Related U.S. Application Data
(60) Provisional application No. 61/047,361, filed on Apr. 23, 2008.

(51) Int. Cl. A61B 18/12; A61B 18/18; H02M 7/5387

(52) U.S. Cl. 606/33; 606/34; 363/132

(57) ABSTRACT

A system for providing power suitable for electrosurgery from a self-contained direct current (DC) energy source according to embodiments of the present invention includes a voltage-affecting circuit having an input and an output, wherein the voltage-affecting circuit is configured to receive energy from the DC energy source at the input and provide boosted DC energy at the output, the boosted DC energy having a voltage greater than a voltage of the DC energy source, and an inverter operable to invert the boosted DC energy to alternating current (AC) energy. The inverter may include a bridge circuit including an arrangement of switches and having an input and an output, wherein the boosted DC energy is received at the bridge circuit input, and a bridge controller operable to control the arrangement of switches to selectively connect the bridge circuit input to the bridge circuit output.

### CROSS-REFERENCE TO RELATED APPLICATIONS

This application claims the benefit of U.S. Provisional Patent Application Ser. No. 61/047,361, filed on Apr. 23, 2008, and entitled, “Control Circuitry for a Tissue Ablation System,” which is incorporated herein by reference in its entirety for all purposes.

### FIELD

Embodiments of the present invention relate generally to surgical instruments, and more specifically to surgical instruments that apply radio frequency (RF) energy to produce surgical effects such as tissue cutting, tissue ablation, hemostasis and others.

### BACKGROUND

The potential applications and recognized advantages of employing electrical energy in surgical procedures continue to increase. For example, electrosurgical techniques are now being widely employed to provide significant localized surgical advantages in open, laparoscopic, and arthroscopic applications, relative to surgical approaches that use mechanical cutting such as scalpels.

Electrosurgical techniques typically entail the use of a hand-held instrument, or pencil, that transfers alternating current (AC) electrical power operating at radio frequency (RF) to tissue at the surgical site. The system also includes a source of RF electrical power, and an electrical return path device, commonly in the form of a return electrode pad attached to the patient away from the surgical site (i.e., a monopolar system configuration) or a smaller return electrode positionable in bodily contact at or immediately adjacent to the surgical site (i.e., a bipolar system configuration). The time-varying voltage produced by the RF electrical power source yields a predetermined electrosurgical effect, such as tissue cutting, coagulation (hemostasis), or ablation.

The process of applying RF electrical power has traditionally employed power supplies called electrosurgical generators. An electrosurgical generator may alternatively be referred to as an electrosurgical unit (ESU) or a Bovie. ESUs typically include means for producing and controlling high voltage (typically over 300 volts and up to about 15,000 volts), high frequency (typically over 100 kHz and up to about 2 MHZ) electrical power using energy supplied through a power cord connected to an external power source, such as a wall power outlet in an operating room or doctor's office. Other power sources include power supplied by vehicles or other portable power supplies.

ESUs are too large to be built into the instruments held by surgeons, and consequently the expensive and bulky ESU needs to be near the patient during the surgical procedure. In addition, the cords connecting the surgical instrument to the ESU can be burdensome while the medical professional is applying RF power to patient tissues. Furthermore, because conventional ESUs require connection to an external power source, isolation circuits may be necessary or desirable.

### SUMMARY

Accordingly, some embodiments of the present invention provide a self-contained RF power source suitable for being incorporated into a surgical instrument that is held by medical clinicians such as surgeons or other physicians and that also provides a means for controlling the application of RF power in an instrument that contains an RF power source. Embodiments of the present invention include a self-contained energy source that produces one or more predetermined surgical effects without a concurrent connection to an external energy source such as line power or power from a vehicle.

A circuit according to embodiments of the present invention can efficiently convert high voltage direct current (DC) into RF current when combined with a compact voltage boosting circuit capable of converting low voltage DC into high voltage DC, optionally with such high voltage DC energy being stored using a high voltage DC storage means. In addition, surgical instruments can be built using such compact RF power supplies by incorporating a self-contained energy source into the instrument along with the aforementioned circuits with the result being surgical instruments that apply RF power to one or more patient tissues to achieve a predetermined surgical effect.

High voltage switching devices, such as bipolar or field effect transistors, can be used in a bridge configuration to replace amplifier circuits and other means being used in existing ESUs to produce high voltage RF power. Using bridge circuits simplifies design, produces a compact design, and leads to higher efficiency, which extends the life of the self-contained energy source. In addition, bridge circuits allow for use of a smaller self-contained power source when a specific amount of energy needs to be delivered to tissue.

Self-contained energy sources include batteries, capacitors, and fuel-cells that can operate at voltage below those needed for electrosurgery and can have their voltages boosted with the high voltage energy stored in capacitors before this high voltage energy is converted to high voltage RF by a DC-to-RF converter such as the previously mentioned bridge circuit.

In short, the inventors have recognized that a self-contained surgical instrument that applies RF power to patient tissues will provide advantages and that such an instrument can be made using circuits different from those now employed to produce medical RF power. Embodiments of the present invention incorporate a self-contained energy source, boost the voltage from the self-contained energy source and store the boosted high voltage energy in a temporary storage means, and convert the stored high voltage energy into RF power suitable for achieving one or more predetermined surgical effects.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates control circuitry for a tissue ablation system, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

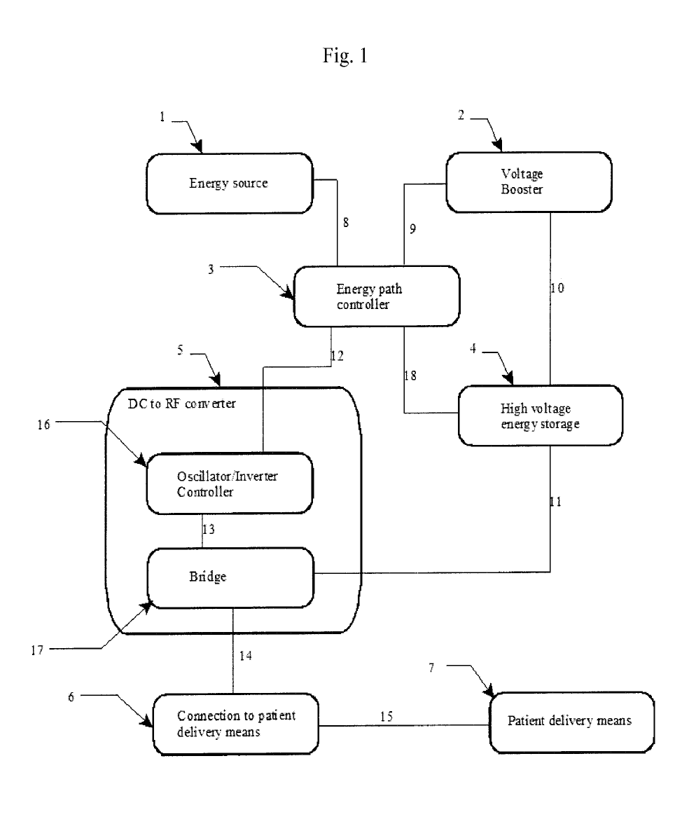

FIG. 1 illustrates one embodiment of the present invention. This embodiment includes an energy source 1, which may be, for example, a self-contained energy source. A self-contained energy source is any energy source that does not use one or more conductive wires to connect to an external energy source, such as line/utility power, while the energy source is delivering power. Examples of self-contained energy sources include, but are not limited to, one or more cells, batteries, fuel cells, capacitors, devices capable of converting electromagnetic radiation into electricity (such as, for example, photovoltaic cells), devices capable of converting mechanical motion into electrical energy, or combinations of one or more types of self-contained energy sources.

Energy source 1 supplies energy to energy booster 2 via energy path controller 3 through one or more conductors 8 and 9. Energy booster 2 increases the voltage from energy source 1 to the voltage desired to achieve the predetermined surgical effect. Such voltage boosting means may include, for example, one or more voltage or current control elements, such as diodes or transistors, and one or more voltage changing elements, such as transformers. According to some embodiments of the present invention, energy booster 2 is a blocking oscillator that includes at least one bipolar transistor, at least one transformer, and at least one diode.

Energy path controller 3 controls when energy is applied from energy source 1 to the various functional units of the circuit, such as energy booster 2. Energy path controller 3 may, for example, disable operation of the device until the user takes some definite action such as moving or removing a mechanical component (not shown) that facilitates safely shipping a device that incorporates the circuit. Energy path controller 3 may include one or more mechanical switches (e.g., button or slide switches) that contain one or more electrical contacts. Energy path controller 3 may also include one or more current or voltage control elements that are controlled by connection to one or more contacts of the mechanical switches. For example, the current or voltage control elements may include diodes, bipolar junction transistors, field effect transistors, and the like. In some embodiments, the mechanical switches control connection of low voltage signals to the control nodes of the electronic current or voltage control elements. Selectable connection of the low voltage signals to the control nodes regulates propagation of high voltage signals through the electronic current or voltage control elements.

The high voltage energy from energy booster 2 flows to high voltage energy storage module 4 through one or more conductors 10. High voltage storage module 4 may be, for example, one or more supercapacitors or other devices capable of storing high voltage electricity.

Energy path controller 3 may include a means for alerting one or more users when a target voltage or other indication of readiness for use has been achieved (for example, when sufficient energy is stored in high voltage energy storage module 4 for electrosurgery). Such indicator means may include, without limitation, visual indicators, such as one or more lights (e.g., light emitting diodes (LEDs), organic LEDs, incandescent lights, photodiodes, etc.), or audible indicators, such as from an acoustic transducer (e.g., piezo audio transducer, speaker, buzzer, etc.), indicating progress toward readiness for use. Energy path controller 3 may interrupt or otherwise control or maintain the voltage or energy storage level of the high voltage energy storage means 4.

The high voltage in high voltage storage module 4 is converted from DC to RF (e.g., a frequency greater than about 2 kHz, or greater than about 10 kHz, or greater than about 50 kHz, or greater than about 100 kHz) by DC-to-RF converter 5. In some embodiments, DC-to-RF converter 5 does not operate at the same time as energy booster 2, in order to facilitate storing energy in high voltage energy storage module 4. For example, energy path controller 3 may control operation using control line 12 such that DC-to-RF converter 5 does not operate at the same time that energy booster 2 is storing energy in high voltage energy storage module 4.

DC-to-RF converter 5 may consist of two functional units, an oscillator/inverter controller 16 and a bridge 17 that are connected using one or more conductive pathways 13. Oscillator/inverter controller 16 generates RF timing pulses that cause one or more voltage or current control elements in bridge 17 to alternately turn on and off to convert DC to current of alternating polarity (e.g. alternating current (AC)). Bridge 17 receives high voltage energy that it inverts from one or more conductive pathways 11. The RF timing pulses from oscillator/inverter controller 16 control both the timing related to when the voltage or current control elements in bridge 17 turn on and off and the amount of time when all of the control elements are off so that no power is flowing. Oscillator/inverter controller 16 may include, for example, integrated circuits used to make pulses, such as 555 timers, operational amplifiers, or discrete components that may be used to make an oscillator such as crystals, ceramic oscillators, transistors, capacitors, or resistors. The oscillator may be of any type including, but not limited to, an astable multivibrator, an Armstrong oscillator, a blocking oscillator, a Clapp oscillator, a Colpitts oscillator, a Hartley oscillator, an oscillistor, a Pierce oscillator, a relaxation oscillator, and RLC circuit, a Royer oscillator, a V\check{a}cká\check{r} oscillator, a Wien bridge oscillator, a virtual cathode oscillator, and the like. Oscillator/inverter controller 16 may also include a combined software, firmware, and/or hardware assembly that is operable to generate timing pulses.

Bridge 17 can be any device that is operable to invert DC energy to AC energy. An example is an H-bridge that uses one or more bipolar junction or field effect transistors. H-bridges are particularly advantageous because of their efficiency, such as when compared to the class C amplifiers typically used in ESUs. Bridge 17 may also be of other types including, but not limited to, a Wheatstone bridge, a Wien bridge, and a Maxwell bridge. Bridge 17 receives one or more control signals from oscillator/inverter controller 16 via one or more conductive pathways 13.

The high voltage RF energy from bridge 17 flows to a connection interface, such as one or more pins of a connector or one or more wires, via one or more conductive pathways 14. The RF energy then flows to patient delivery means 7 via one or more conductive pathways 15. Patient delivery means 7 may include one or more conductors. More particularly, patient delivery means 7 may be a monopolar device in which a small active electrode is in the surgical instrument held by, for example, a surgeon, and a larger return electrode interfaces with the patient. Patient delivery means 7 may also be in a bipolar configuration in which both the source and sink electrodes (commonly called active and return) are in the instrument held by a surgeon or other medical professional.

In one embodiment the circuit, the energy supply, and the means for conveying RF energy to the patient are in a single device such that it is a unified assembly in a single package. Such a device may be a single use device or a device that may be reused. The device in which the control circuit is included may be any type of device suitable for use in electrosurgery, according to embodiments of the present invention. For example, the control circuit described may be integrated into a tendon cauterizing and cutting device.

Circuits according to embodiments of the present invention convert low voltage direct current (DC) (e.g., less than about 50 volts or less than about 16 volts), from a self-contained energy source such as batteries, capacitors, or fuel cells, into radio frequency (RF) power at high voltage (e.g., voltages exceeding at least about 225 volts, or at least about 280 volts, or at least about 300 to 350 volts) with such RF power being delivered to one or more patient tissues to achieve at least one predetermined surgical effect such as cutting, coagulation (hemostasis), or ablation. The circuits may include a voltage boost means to increase the voltage from the energy source, a high voltage energy storage means, a means to convert high voltage DC energy to RF energy, a means to connect the RF energy to a means that delivers energy to one or more patient tissues, and may also contain a means to deliver energy to the patient, such as one or more elements that transfer energy using at least one of conduction or capacitive coupling. The circuits may include a means to control transfer of energy from the energy source or the high voltage energy storage such as one or more mechanical switches with one or more electrical contacts. Such mechanical switches may control the flow of electrical energy directly or by controlling, either directly or indirectly, electronic switches such as bipolar or field effect transistors.

Thus, in one embodiment of the present invention, a system providing power suitable for electrosurgery from a self-contained direct current (DC) energy source includes: a voltage-affecting circuit having an input and an output, wherein the voltage-affecting circuit is configured to receive energy from the DC energy source at the input and provide boosted DC energy at the output having a voltage greater than a voltage of the DC energy source; and an inverter operable to invert the boosted DC energy to alternating current (AC) energy. The inverter may have any form suitable for inverting DC energy to AC energy. For example, in some embodiments the inverter includes a bridge circuit having an arrangement of switches between an input (which receives the boosted DC energy) and an output, and a bridge controller operable to control the plurality of switches to selectively connect the bridge circuit input to the bridge circuit output.

Embodiments of the present invention may also include an electrical energy storage module configured to store the boosted DC energy. An energy path controller may also be incorporated to control energy flow between the DC energy source, voltage-affecting circuit, inverter, and electrical energy storage module.

In another embodiment of the present invention, an electrosurgical device includes: an energy source coupling configured to receive a self-contained direct current (DC) energy source; a voltage-affecting circuit having an input and an output, wherein the voltage-affecting circuit is connected to the power source coupling to receive energy from the DC energy source at the input and provide boosted DC energy at the output having a voltage greater than a voltage of the DC energy source; an inverter operable to invert the boosted DC energy to alternating current (AC) energy; and an energy delivery assembly configured to deliver the AC energy to a patient.

The following U.S. Patents are incorporated herein by reference:

7,326,201; 7,310,545; 7,288,092; 7,237,555; 7,226,448; 7,201,749;
7,083,614; 6,960,206; 6,907,297; 6,849,075; 6,736,808; 6,695,838;
6,635,054; 6,616,655; 6,607,520; 6,358,247; 6,143,019; 6,086,582;
6,016,809; 5,980,516; 5,964,753; 5,881,727; 5,824,005; 5,590,657;
5,573,533; 5,431,649; 5,423,807; 5,383,922; 5,083,565; 5,047,026.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A system for providing power suitable for electrosurgery from a self-contained direct current (DC) energy source, the system comprising:
a voltage-affecting circuit having an input and an output, wherein the voltage-affecting circuit is configured to receive energy from the DC energy source at the input and provide boosted DC energy at the output, the boosted DC energy having a voltage greater than a voltage of the DC energy source; and
an inverter operable to invert the boosted DC energy to alternating current (AC) energy.

2. The system of claim 1, wherein the inverter comprises:
a bridge circuit including an arrangement of switches and having an input and an output, wherein the boosted DC energy is received at the bridge circuit input; and
a bridge controller operable to control the arrangement of switches to selectively connect the bridge circuit input to the bridge circuit output.

3. The system of claim 1, further comprising:
an electrical energy storage module configured to store the boosted DC energy.

4. The system of claim 3, further comprising:
an energy path controller operable to control energy flow between the DC energy source, the voltage-affecting circuit, the inverter, and the electrical energy storage module.

5. The system of claim 1, wherein the DC energy source, the voltage-affecting circuit, and the inverter are housed in a hand-held surgical instrument.

6. The system of claim 1, wherein the voltage-affecting circuit comprises a blocking oscillator circuit.

7. The system of claim 1, wherein the AC energy is radio frequency (RF) energy.

8. An electrosurgical device comprising:
an energy source coupling configured to receive a self-contained direct current (DC) energy source;
a voltage-affecting circuit having an input and an output, wherein the voltage-affecting circuit is connected to the power source coupling to receive energy from the DC energy source at the input and provide boosted DC energy at the output having a voltage greater than a voltage of the DC energy source;
an inverter operable to invert the boosted DC energy to alternating current (AC) energy; and
an energy delivery assembly configured to deliver the AC energy to a patient.

9. The electrosurgical device of claim 8, wherein the inverter comprises:
a bridge circuit including an arrangement of switches and having an input and an output, wherein the boosted DC energy is received at the bridge circuit input; and
a bridge controller operable to control the arrangement of switches to selectively connect the bridge circuit input to the bridge circuit output.

10. The electrosurgical device of claim 8, further comprising:
an electrical energy storage module configured to store the boosted DC energy.

11. The electrosurgical device of claim 8, further comprising:
an energy path controller operable to control energy flow between the DC energy source, voltage-affecting circuit, inverter, and electrical energy storage module.

12. The electrosurgical device of claim 8, wherein the DC energy source, the voltage-affecting circuit, and the inverter are housed in the electrosurgical device.

13. The electrosurgical device of claim 8, wherein the voltage-affecting circuit comprises a blocking oscillator circuit.

14. The electrosurgical device of claim 8, wherein the AC energy is radio frequency (RF) energy.

15. A method for delivering power from a self-contained energy source in a surgical instrument, the method comprising:
receiving energy from the self-contained energy source;
boosting the energy from the self-contained energy source to a boosted energy greater than the energy source;
storing the boosted energy in a storage module;
converting the boosted energy from a direct current (DC) boosted energy into a radio frequency (RF) boosted energy; and
delivering the RF boosted energy to a patient.

16. The method of claim 15, further comprising:
controlling an energy path between the self-contained energy source, an energy booster, the storage module, and a DC to RF converter.

17. The method of claim 15, wherein converting the boosted energy from the DC boosted energy into the RF boosted energy comprises passing the DC boosted energy through an inverter circuit and a bridge circuit.

18. The method of claim 17, wherein the bridge circuit includes an arrangement of switches and has an input and an output, wherein the DC boosted energy is received at the bridge circuit input, and wherein the bridge circuit further includes a bridge controller operable to control the arrangement of switches to selectively connect the bridge circuit input to the bridge circuit output.
